(19) United States
(12) Patent Application Publication
(10) Pub. No.: US 2023/0404392 A1
(43) Pub. Date: Dec. 21, 2023

(54) CATARACT DETECTION WITH AN AUTO-REFRACTOMETER

(71) Applicant: iCRx, Inc., Tucson, AZ (US)

(72) Inventors: Pierre A. Blanche, Tucson, AZ (US); Erol Ozgur, Tucson, AZ (US); Ram Voorakaranam, Tucson, AZ (US)

(21) Appl. No.: 17/843,763

(22) Filed: Jun. 17, 2022

(51) Int. Cl. A61B 3/103; A61B 3/117; A61B 3/10

(52) U.S. Cl. CPC A61B 3/103; A61B 3/1176; A61B 3/1015

(57) ABSTRACT

The present invention is directed to an automated ophthalmic cataract detection using an auto-refractometer. The present invention features a method for early identification of cataracts in a patient based on spot pattern intensity. In some embodiments, the method may comprise providing an auto-refractometer comprising a Shack-Hartman sensor, and an intensity detector. The method may further comprise providing a computing device, measuring, by the Shack-Hartman sensor, a spot pattern from an eye of the patient, measuring, by the intensity detector, an intensity homogeneity value of the spot pattern, and detecting, by the computing device, a cataract in the eye of the patient based on the intensity homogeneity value. The computing device may detect the cataract if the intensity homogeneity ratio is below a threshold value.

### FIELD OF THE INVENTION

The present invention is directed to an automated ophthalmic cataract detection using an auto-refractometer.

### BACKGROUND OF THE INVENTION

As defined for the purpose of this invention, an auto-refractometer is an instrument capable of measuring the ophthalmic aberration of an eye. To do so, the auto-refractometer sends a collimated beam of infrared light to the eye of the subject and measures the wavefront of the beam reflected by the retina. This wavefront measurement is done with a wavefront sensor such as a Shack-Hartmann sensor.

A Shack-Hartmann sensor is a wavefront sensor that uses a microlens array in front of a 2D detector such as a CCD or CMOS. The lens array is focusing the incoming beam at the focal distance of each lens. The position and size of the different focal spots are used to determine the wavefront curvature that can be further translated into Zernike coefficients and/or CSA ophthalmic aberrations.

In its prior art form, neither the Shack-Hartmann sensor nor the auto-refractometer use the intensity of the light coming back from the retina of the subject.

In ophthalmology, a cataract is an affection resulting from the cloudy area in the intraocular lens of the eye that leads to a decrease in vision. There are different stages of cataracts from early to advanced, and from localized to general. Thus, there exists a present need for a method of early automated cataract detection through the use of an auto-refractometer.

### BRIEF SUMMARY OF THE INVENTION

It is an objective of the present invention to provide methods and systems that allow for automated ophthalmic cataract detection using an auto-refractometer, as specified in the independent claims. Embodiments of the invention are given in the dependent claims. Embodiments of the present invention can be freely combined with each other if they are not mutually exclusive.

The present invention features a method for early identification of cataracts (e.g. before a patient's complaints begin) in a patient based on spot pattern intensity. In some embodiments, the method may comprise providing an auto-refractometer comprising a Shack-Hartman sensor, and an intensity detector. The method may further comprise providing a computing device, measuring, by the Shack-Hartman sensor, a spot pattern from an eye of the patient, measuring, by the intensity detector, an intensity homogeneity value of the spot pattern, and detecting, by the computing device, a cataract in the eye of the patient based on the intensity homogeneity value. The computing device may detect the cataract if the intensity homogeneity ratio is below a threshold value.

The present invention features a system for early identification of cataracts in a patient based on spot pattern intensity. In some embodiments, the system may comprise an auto-refractometer comprising a Shack-Hartman sensor, and an intensity detector. The system may further comprise a computing device communicatively coupled to the auto-refractometer. The computing device may be capable of measuring, by the Shack-Hartman sensor, a spot pattern from an eye of the patient, measuring, by the intensity detector, an intensity homogeneity value of the spot pattern, calculating, by the intensity detector, a signal-to-noise ratio based on the intensity homogeneity value, and detecting, by the computing device, a cataract in the eye of the patient based on the intensity homogeneity value and the signal-to-noise ratio. The computing device may detect the cataract if the intensity homogeneity ratio is below a threshold value.

One of the unique and inventive technical features of the present invention is the measurement of intensity changes in an eyeball spot pattern for cataract detection. Without wishing to limit the invention to any theory or mechanism, it is believed that the technical feature of the present invention advantageously provides for early and efficient detection of cataracts in a patient's eye, thus allowing for earlier treatment of this disorder. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWINGS

The features and advantages of the present invention will become apparent from a consideration of the following detailed description presented in connection with the accompanying drawings in which:

FIG. 1 shows a flow chart of a method for early identification of cataracts in a patient based on spot pattern intensity.

FIG. 2 shows a schematic of a system for early identification of cataracts in a patient based on spot pattern intensity.

### DETAILED DESCRIPTION OF THE INVENTION

Following is a list of elements corresponding to a particular element referred to herein:
100 system
110 auto-refractometer
111 Shack-Hartman sensor
112 intensity detector
120 computing device

A difference in the spot intensity for the same patient between two tests over a time, or a specific loss of spot pattern intensity on a particular location on the spot images can be interpreted as there is a loss in the transparency in the cornea, which is one of the main symptoms of cataract.

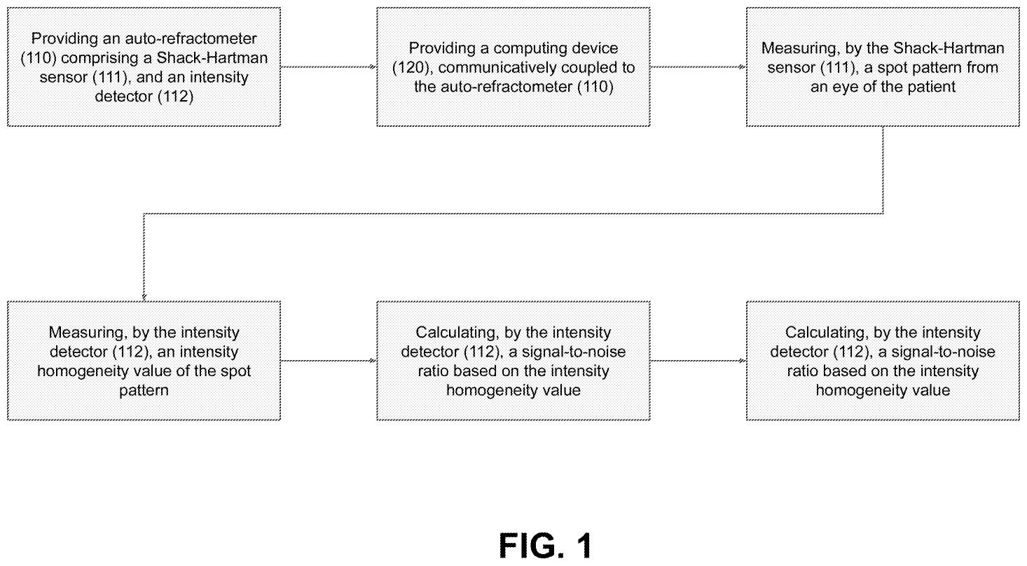

Referring now to FIG. 1, the present invention features a method for early identification of cataracts in a patient based on spot pattern intensity. In some embodiments, the method may comprise providing an auto-refractometer (110) comprising a wavefront sensor (111) configured to measure intensity of the wavefront at a plurality of points. The method may further comprise providing a computing device (120), communicatively coupled to the auto-refractometer (110), comprising a processor capable of executing computer-readable instructions, and a memory component comprising a plurality of computer-readable instructions. The method may further comprise measuring, by the wavefront sensor (111), a spot pattern from an eye of the patient, measuring, by the wavefront sensor (111), an intensity homogeneity value of the spot pattern, calculating, by the wavefront sensor (111), a signal-to-noise ratio based on the intensity homogeneity value, and detecting, by the computing device (120), a cataract in the eye of the patient based on the intensity homogeneity value. The computing device (120) may detect the cataract if the intensity homogeneity ratio is below a threshold value. In some embodiments, the threshold value may be 50% to 99%. In other embodiments, the threshold value may be 75% to 99%. In other embodiments, the threshold value may be 90%. In some embodiments, the computing device (120) may detect the cataract if the signal-to-noise ratio is low. In some embodiments, the computing device (120) may detect the cataract if the signal-to-noise ratio requires a high gain and low threshold to detect.

The present invention features a method for early identification of cataracts in a patient based on spot pattern intensity. In some embodiments, the method may comprise providing an auto-refractometer (110) comprising a Shack-Hartman sensor (111), and an intensity detector (112). The method may further comprise providing a computing device (120), measuring, by the Shack-Hartman sensor (111), a spot pattern from an eye of the patient, measuring, by the intensity detector (112), an intensity homogeneity value of the spot pattern, and detecting, by the computing device (120), a cataract in the eye of the patient based on the intensity homogeneity value. The computing device (120) may detect the cataract if the intensity homogeneity ratio is below a threshold value. In some embodiments, the threshold value may be 50% to 99%. In other embodiments, the threshold value may be 75% to 99%. In other embodiments, the threshold value may be 90%. In some embodiments, the computing device (120) may detect the cataract if the signal-to-noise ratio is low. In some embodiments, the computing device (120) may detect the cataract if the signal-to-noise ratio requires a high gain and low threshold to detect.

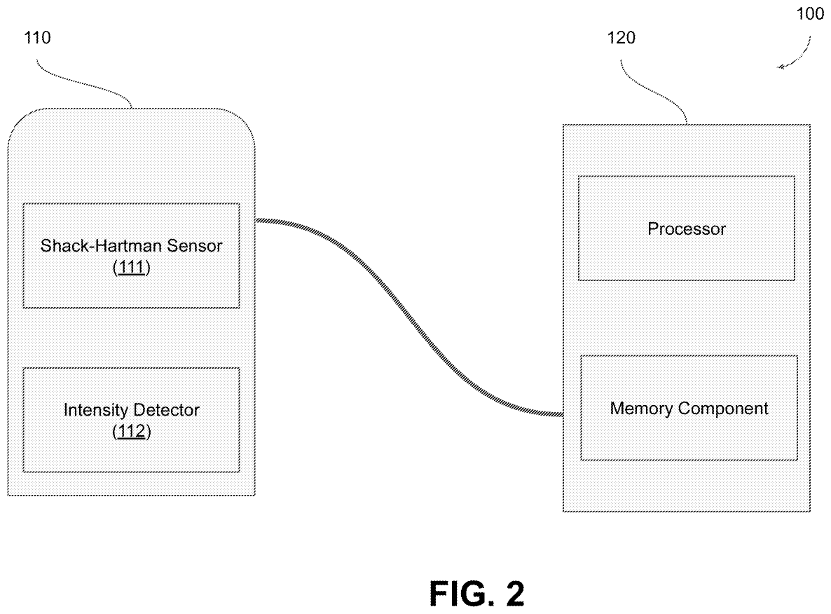

Referring now to FIG. 2, the present invention features a system (100) for early identification of cataracts in a patient based on spot pattern intensity. In some embodiments, the system (100) may comprise an auto-refractometer (110) comprising a Shack-Hartman sensor (111), and an intensity detector (112). The system (100) may further comprise a computing device (120) communicatively coupled to the auto-refractometer (110), comprising a processor capable of executing computer-readable instructions, and a memory component comprising computer-readable instructions. The computer-readable instructions may comprise measuring, by the Shack-Hartman sensor (111), a spot pattern from an eye of the patient, measuring, by the intensity detector (112), an intensity homogeneity value of the spot pattern, calculating, by the intensity detector (112), a signal-to-noise ratio based on the intensity homogeneity value, and detecting, by the computing device (120), a cataract in the eye of the patient based on the intensity homogeneity value and the signal-to-noise ratio. The computing device (120) may detect the cataract if the intensity homogeneity ratio is below a threshold value. In some embodiments, the threshold value may be 50% to 99%. In other embodiments, the threshold value may be 75% to 99%. In other embodiments, the threshold value may be 90%. In some embodiments, the computing device (120) may detect the cataract if the signal-to-noise ratio is low. In some embodiments, the computing device (120) may detect the cataract if the signal-to-noise ratio requires a high gain and low threshold to detect.

The present invention uses the intensity of the spot pattern generated on the Shack-Hartmann sensor of an auto-refractometer to diagnose cataracts.

On an eye without cataracts, the intensity of the different sports forming on the Shack-Hartman sensor is quite homogeneous. The intensity homogeneity is within 90% for all the points. Similarly, the SNR is high, not requiring a high gain and/or low threshold value to detect. This is explained by the fact that the reflection from the retina, and passing through the subject's intraocular lens does not suffer from scattering or localized absorption.

Intensity homogeneity means that the signal intensity from individual spots have a distribution with a homogeneity of around 90%. If it is less homogenous, this may be interpreted as a sign of local transparency loss on the eye. The signal-to-noise, on the other hand, is compensated by automated gain and threshold adjustment, and if a high gain and low threshold is required to observe the spot patterns, and if this was not the case in the previous measurement of the same patient a time ago, this is considered as a progressive loss in the transparency of the eye.

On an eye with cataract, the intraocular lens adds some scattering to the light beam reflected by the retina, as well as some absorption. The absorption lowers the signal, and the scattering increases the background noise, such that lower SNR is observed. “Lower SNR” may be defined as a SNR that requires a high gain (at least 30 dB) and low threshold (5 in 8-bit data) to detect. Also, when the cataract is localized to some specific regions of the lens, the intensity of the different focal points observed at the Shack-Hartmann sensor becomes inhomogeneous (homogeneity below 90%).

Therefore, the value of the homogeneity, as well as the SNR of the focal points at a Shack-Hartmann sensor of an auto-refractometer, can be used to detect cataracts in a subject.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A method for early identification of cataracts in a patient based on spot pattern intensity, the method comprising:
a. providing an auto-refractometer (110) comprising:
i. a wavefront sensor (111) configured to measure intensity of the wavefront at a plurality of points;
b. providing a computing device (120), communicatively coupled to the auto-refractometer (110), comprising a processor capable of executing computer-readable instructions, and a memory component comprising a plurality of computer-readable instructions;
c. measuring, by the wavefront sensor (111), a spot pattern from an eye of the patient;
d. calculating an intensity homogeneity value of the spot pattern;
e. calculating a signal-to-noise ratio based on the intensity homogeneity value; and
f. identifying, by the computing device (120), a cataract in the eye of the patient based on the intensity homogeneity value and the signal-to-noise ratio;
wherein the computing device (120) identifies the cataract if the intensity homogeneity ratio is below a threshold value.

2. The method of claim 1, wherein the threshold value is 75% to 99%.

3. The method of claim 2, wherein the threshold value is 90%.

4. The method of claim 1, wherein the computing device (120) detects the cataract if the signal-to-noise ratio is low.

5. The method of claim 4, wherein the computing device (120) detects the cataract if the signal-to-noise ratio requires a high gain and low threshold to detect.

6. A method for early identification of cataracts in a patient based on spot pattern intensity, the method comprising:
a. providing an auto-refractometer (110) comprising:
i. a Shack-Hartman sensor (111), and
ii. an intensity detector (112);
b. providing a computing device (120);
c. measuring, by the Shack-Hartman sensor (111), a spot pattern from an eye of the patient;
d. measuring, by the intensity detector (112), an intensity homogeneity value of the spot pattern; and
e. detecting, by the computing device (120), a cataract in the eye of the patient based on the intensity homogeneity value;
wherein the computing device (120) detects the cataract if the intensity homogeneity ratio is below a threshold value.

7. The method of claim 6, wherein the threshold value is 75% to 99%.

8. The method of claim 7, wherein the threshold value is 90%.

9. The method of claim 6, wherein the computing device (120) detects the cataract if the signal-to-noise ratio is low.

10. The method of claim 9, wherein the computing device (120) detects the cataract if the signal-to-noise ratio requires a high gain and low threshold to detect.

11. A system (100) for early identification of cataracts in a patient based on spot pattern intensity, the system (100) comprising:
a. an auto-refractometer (110) comprising:
i. a Shack-Hartman sensor (111), and
ii. an intensity detector (112); and
b. a computing device (120) communicatively coupled to the auto-refractometer (110), comprising a processor capable of executing computer-readable instructions, and a memory component comprising computer-readable instructions for:
i. measuring, by the Shack-Hartman sensor (111), a spot pattern from an eye of the patient;
ii. measuring, by the intensity detector (112), an intensity homogeneity value of the spot pattern;
iii. calculating, by the intensity detector (112), a signal-to-noise ratio based on the intensity homogeneity value; and
iv. detecting, by the computing device (120), a cataract in the eye of the patient based on the intensity homogeneity value and the signal-to-noise ratio;
wherein the computing device (120) detects the cataract if the intensity homogeneity ratio is below a threshold value.

12. The system (100) of claim 11, wherein the threshold value is 75% to 99%.

13. The system (100) of claim 12, wherein the threshold value is 90%.

14. The system (100) of claim 11, wherein the computing device (120) detects the cataract if the signal-to-noise ratio is low.

15. The system (100) of claim 14, wherein the computing device (120) detects the cataract if the signal-to-noise ratio requires a high gain and low threshold to detect.
